(19) United States
(12) Patent Application Publication
(10) Pub. No.: US 2009/0012387 A1
(43) Pub. Date: Jan. 8, 2009

(54) ENCODING AND TRANSMISSION OF SIGNALS AS RF SIGNALS FOR DETECTION USING AN MR APPARATUS

(75) Inventors: Lars Peter Gruner Hanson, Copenhagen (DK); Torben Ellegard Lund, Copenhagen (DK); Christian Georg Gruner Hanson, Glostrup (DK)

Correspondence Address: KNOBBE MARTENS OLSON & BEAR LLP, 2040 MAIN STREET, FOURTEENTH FLOOR, IRVINE, CA, 92614, US

(73) Assignee: HVIDOVRE HOSPITAL, Hvidovre (DK)

(21) Appl. No.: 11/597,577

(22) PCT Filed: May 25, 2005
(86) PCT No.: PCT/DK2005/000343

§ 371 Date: Mar. 18, 2008

(30) Foreign Application Priority Data
May 25, 2004 (DK) PA 2004 00818

(51) Int. Cl. A61B 5/055; G01R 33/48

(52) U.S. Cl. 600/411; 324/307

(57) ABSTRACT

The invention provides a novel way of handling electric or electromagnetic signals during magnetic resonance (MR) measurements. Non-MR data signals such as EPH signals (e.g. EEG, ECG, blood pressure, respiration) or subject responses (e.g. keystrokes, joystick movements) originating in the MR suite is recorded while performing magnetic resonance imaging or spectroscopy. Relatively simple, possibly battery driven hardware is used to transform the non-MR signals into radio waves detectable by the MR apparatus. The electrical signals are in this way encoded as artifacts appearing in the MR images or spectra outside the region of interest, and the encoded signals can subsequently be reconstructed from the signal recorded by the scanner If oversampling is employed, artifacts can be avoided altogether. The method inherently provides superior synchronisation between the sampling of non-MR data signals and the MR sequence. The invention minimises the need for costly special MR adapted equipment and can be applied with scanners for MR imaging as well as with NMR spectrometers.

### FIELD OF THE INVENTION

The invention relates to the handling of electric and electromagnetic signals inside a room with a nuclear magnetic resonance (MR) apparatus such as a scanner or a spectrometer. In particular, the invention relates to recording of physiological signals from a patient during scanning.

### BACKGROUND OF THE INVENTION

Sometimes, it is necessary to acquire additional data during magnetic resonance scanning or spectroscopy, typically in the form of non-MR electric or electromagnetic (EM) signals. These data range from electrophysiological (EPH) signals such as physiological time courses (e.g. electroencephalograms (EEG), electrocardiograms (ECG), blood pressure, respiration etc) to subject responses (e.g. keystrokes, joystick movements). As will be described below, several issues complicate the acquisition of such additional data in the environment of a MR apparatus.

There exist examples of acquisition of radio frequency (RF) signals induced by the field of the MR apparatus that indicate values of local parameters in the subject or object. Here, a quartz crystal is used as a sensor for the local RF field strength (Wang and Leigh, Magn. Reson. Med. 33 (1995), p. 843) or for the local temperature (Wang and Leigh, 3 magn. reson. B, 105 (1994), p. 25 and Simon, J. magn. reson. 128 (1997), p. 194). Both applications make use of the excitation of resonance frequencies in quartz crystals by the RF excitation pulses from a MR apparatus. The excited resonance signals can subsequently be detected by the coils of the MR apparatus. The amplitude of the resonance signal is proportional to the field strength of the RF excitation pulses and provides a measure of the field strength from the MR apparatus in the quartz crystal. The frequency of the quartz resonances varies with the temperature the crystal and the frequency of the resonance signals thus provides a measure of the temperature of the quartz crystal.

In the field and temperature sensors described in the above, the quartz resonance signal is not used to convey data in the form of signals or messages; the signal only reflects the local environment inside the crystal. To convey additional data such as a data sequence or a message, this does not suffice.

Several issues complicate the acquisition of this additional data in the faraday cage of an MR spectrometer or an MR imaging (MRI) suite. First, the additional non-MR apparatus in the scanner room may create unwanted distortions in the MR-images, known as image artifacts. As a result, the sampling equipment needs to be MR compatible, meaning safe, well-functioning at high field, insensitive to radio frequency waves and electromagnetically silent at MR frequencies. Getting the acquired additional signal out of the scanner room is a second problem. This either requires fibre optics or filters that can let the signal out while keeping the Faraday cage intact. A third problem has to do with the MR-scanner disturbing the acquired signal by generating EM noise in the signals, referred to as signal artifacts. Recording of electrophysiological signals during MRI poses extra difficulty as the rapidly changing magnetic fields induce electrode voltages that are often orders of magnitude stronger than the weak electrical signals originating from neural activity. This noise is in particular generated during the fast changes in magnetic gradients needed for MR-scanning. Traditionally EPH-recordings have therefore been performed in silent periods during the scan, where images were not acquired. However, functional MRI (fMRI) mostly used to measure brain function, for example, benefits from imaging with high temporal resolution, and unnecessary pauses should normally be avoided.

Recording of EPH signals and subject responses may for example represent responses to stimuli presented to a patient in the scanner. They often need to be correlated with MR imaging or spectroscopy, thus adding the complexity of synchronization between MR data and non-MR data. When the data are to be analysed, a fifth problem arises. The MR and additional data are stored in different equipment (e.g. on one computer per extra signal type), making data storage, recalling and correlation troublesome.

A particularly interesting and demanding example is recording of electroencephalography (EEG) during fMRI, see e.g. U.S. Pat. No. 5,445,162 and Ives et al., Electroencephalography and Clinical Neurophysiology, 87, 417 (1993). This combination has great potential ranging from patient supervision, over research in basic neurological processes to improved diagnosis of epilepsy. However, EEG recording in an MRI environment suffers extreme degradation due to pulse and imaging artifacts that are often orders of magnitude larger than the EEG signal of interest. The imaging artifacts are induced by the strong and rapidly changing gradients used for fast imaging. The corresponding artifacts can be reduced somewhat by methods limiting the presence of current loops, e.g. by use of special montages and a common reference or dual-lead electrodes, lead-twisting and stable support of the electrodes. In U.S. Pat. No. 5,445,162 and Ives et al., a careful selection and arrangement of analogue multiplexed cable-telemetry equipment has been used to eliminate ferrous materials and RF sources in the EEG equipment.

Filtering techniques, e.g, moving average template subtraction followed by adaptive noise reduction, have also been used to reduce the pulse, motion and imaging artifacts to acceptable levels, but there is a limit to the precision determined by the shot-to-shot variation in the distortion. Furthermore, removal of large noise components from small signals is demanding in terms of bandwidth, timing control, digital resolution and linearity. As distortions and their variation increase with field/gradient strength, imaging speed and patient motion, moving average filtering alone does not provide satisfactory EEGs in all situations.

These problems also pertain to the EEG recording method disclosed by Cohen in US 2004/0097802 involving the synchronization of high-performance electrophysiological equipment with the MR scanner using a clock signal from the scanner. Using the achieved, very precise synchronization, the repetitive gradient noise from the scanner can be estimated and subtracted.

An alternative “stepping stone sampling” technique of particular interest in the present context was introduced by Anami et al (Neuroimage 19, 281), who demonstrated that EEG can be recorded with limited distortion in the short periods between gradient reversals during echo-planar imaging (EPI). The method benefits from synchronization between scanner and EEG clocks. Triggered 20 kHz sampling was used to record the EEG during 400 μs silent periods where also the MR signals were measured, i.e., in the periods between changes of directions of k-space-traversal in blipped EPI. This method is highly suited for fMRI.

The setup used by Anami et al. is fairly complicated, and relies on microsecond synchronization between scanner and EEG equipment obtained by driving the EEG-system with the scanner clock and using high bandwidth sampling and triggered sample-hold. Also, the MR sequences generally have to be edited to provide the needed triggering. After acquisition, the problem remains of correlating EEG and MR images stored and time-stamped differently in separate computer systems.

### SUMMARY OF THE INVENTION

It is an object of the invention to provide a method and a system for recording electric and electromagnetic non-MR data signals using a MR apparatus.

It is another object of the invention to provide a method and a system for synchronising the recording of MR data signals and non-MR data signals in the MR apparatus.

It is yet another object of the invention to provide a method and a system for easing the recording of electrophysiological (EPH) signals from a scanned subject during scanning.

The problems related to artifacts in MR data signals and non-MR data signals are present in both MR scanners and NMR spectrometers, although they relate to very different fields. In the present description, the term MR apparatus covers both MR scanners used for imaging, NMR spectrometers used for spectroscopy and any other application or apparatus for exciting and detecting precessing nuclear spins. In the present specification, the term MR apparatus designates an apparatus having several parts, some typically situated inside the faraday cage, some typically situated outside. The MR apparatus includes at least the following parts:
A scanner, being the power supplies and coils generating the static and varying magnetic and RF signals. The subject or object resides in the strong fields inside the faraday cage.
A part receiving signals from precessing nuclear spins and adapting the received signal to be transmitted to the succeeding part of the MR apparatus. This part resides inside the faraday cage and may also be referred to as receiving coils or antennas.
A part converting the MR data signals to digital signals, this part may reside inside or outside the faraday cage.
A part which stores the digital MR data signals, this may be situated close to or far away from the faraday cage.
Parts which transmit various MR data signals between the above parts, and which may include filters, modulators, multiplexers etc. for manipulating the MR data signals in course of transit. These parts are situated both inside and outside the faraday cage.

Also, in the present context, a MR signal or MR data signal is any signal which is generated by precessing nuclear spins and recorded by a receiving coil as well as signals derived from the recorded signal, e.g. by sampling, modulation, multiplexing, transformation, conversion, etc. The signal path for a MR signal in the MR apparatus starts at the receiving coils of the MR apparatus and ultimately ends at the storage medium holding the stored MR data. The signal carrying the MR data may thus be manipulated, filtered, converted etc. several times along the MR signal path.

The present invention attains the objects mentioned above and other objects by exploiting MR apparatuses capabilities of handling signals in the radio frequency (RF) range. As MR signals from the precessing nuclear spins are relatively weak RF signals, MR apparatus are equipped with high quality components for fast and extremely precise sampling and recording of RF signals.

By intentionally modulating other signals holding non-MR data onto a signal having a frequency in the range of one or more channels of the MR apparatus MR apparatus, the resulting signal can be controllably received by the MR apparatus. If this signal is an RF signal received by the receiving coils of the MR apparatus, it will generate a large but controlled (image) artifact in the recorded MR data. By selecting a proper frequency range and modulation scheme for the modulation onto the RF signal, the artifact from these signals will not interfere with the recorded MR data. The non-MR data signals may e.g. be modulated onto an RF carrier with a frequency corresponding to the edge of an image or to a position in a spectrum that is not typically used for identification. The resulting artifact is thereby recorded and saved in the MR apparatus and may be restored at any time. Preferably, the non-MR data signal is amplitude modulated onto a carrier signal, however, frequency- or phase modulation may be applied as well as multiplexing of several non-MR data signals on the same carrier. Also, modulation is the use of a data signal to actively and deliberately modify or adjust another signal referred to as a carrier (typically of higher frequency) so that the data signal can be restored later by demodulation.

In the present context, non-MR data or non-MR data signal is any signal which is not directly generated or induced by the MR apparatus. The non-MR data signals are signals from another apparatus or devices separate from the MR apparatus. In order to be recorded by the MR scanner as described in the above, the non-MR data signals are modulated onto a carrier signal in the RF band of the MR apparatus, and the modulated carrier signal is then transmitted to and recorded by the MR apparatus. The signals modulated onto the RF carrier may e.g. be EPH signals such as EEG or ECG signals, signals resulting from monitoring of blood pressure, respiration etc., or signals representing subject responses such as keystrokes, joystick movements or speech.

Thermal, optical and other non-electrical signal do not suffer from the same limitations inside a MR suite as electrical signals do. For this reason, the non-MR data signal is preferably an electric signal, such as a signal resulting from a measurement of a value or the detection of an event using an electrical apparatus separate from the MR apparatus.

Signals from precessing nuclear spins in the scanned subject or object, which are induced directly by the magnetic and electromagnetic fields from the MR apparatus are not non-MR data signals according to the above definition. However, these signals may be used as the carrier signals onto which the non-MR data signal may be modulated. In this case, an apparatus or a device separate from the MR apparatus may use the non-MR data signal to modulate the magnetic or RF fields in a local region in the MR receiver coil so that the non-MR data is encoded in RF waves emitted from the region at the Larmor frequency.

Hence, there are three types of signals to distinguish between in the present context;
1. Non-MR data signals which may be any type of signal generated by an apparatus or device separate from the MR apparatus. This type of signals are also referred to as simply non-MR data or non-MR signal;
2. RF carrier signals modulated with non-MR data signals, transmitted to the MR apparatus wirelessly or by wired connection. The carrier signals may be generated by a frequency generator or an oscillator, or may be MR signals from regions where the RF field from the MR apparatus has been modulated with the non-MR data signals. This type of signal will be referred to as non-MR data modulated signals or simply modulated signals;
3. RF signals from precessing nuclear spins, which are not modulated with non-MR data signals. These correspond to normal MR signals and will be referred to as such.

Unless a distinction is required by the circumstances, both the non-MR data signals (type 1) and the non-MR data modulated RF carrier signals (type 2) will be collectively referred to as non-MR data signals as the first is often contained in the second by modulation. Also, when it is clear from the context, these signals will also be referred to simply as electric or EM signals.

It is an advantage of the invention that the recording of non-MR data signals during a MR sequence does not lead to uncontrollable artifacts on recorded MR data signals and vice versa. As the non-MR data are recorded without any dedicated equipment, the invention minimises the need for costly special equipment adapted for use in MR environments. Also, no new signal lines are needed for transferring data out of the faraday cage or scanner room.

Since the MR apparatus performs the recording of the non-MR data signals, the recording of non-MR data signals is inherently synchronized with the MR measuring sequence. This provides the advantage of reducing artifacts on the non-MR data signals created by the changes in magnetic gradients from the MR scanner. Also it facilitates the subtraction of residual gradient artifacts.

It is an added bonus that the non-MR data corresponding to a MR measuring sequence is saved together and in synchronisation with the MR data from the sequence, which simplifies the task of keeping track of files and synchronisation.

According to a first aspect, the present invention provides a method for generating, transmitting and recording a non-MR data signal during a magnetic resonance sequence, the method comprising the steps of:
in a magnetic resonance apparatus room, generating a non-MR data signal and modulating the non-MR data signal onto a magnetic, electric or electromagnetic signal to form a modulated carrier signal having a frequency in the range of one or more channels of the magnetic resonance apparatus,
introducing the modulated carrier signal into a signal path for MR signals of a MR-apparatus before an analogue to digital conversion of MR signals, and
demodulating the introduced modulated carrier signal to restore the non-MR data signal or a function thereof.

Preferably, the step of modulating the non-MR data comprises modulating the non-MR data to an electric or EM carrier signal having a predetermined frequency in the RF band. The carrier signal is typically provided by a clock signal generator or an oscillator in a dedicated modulator. The frequency of the carrier signal is important since it differentiates the non-MR data signals from the MR data signals and thereby allows restoration of the non-MR data even though the signals have been handled together by the MR apparatus. Hence, the predetermined frequency is preferably distinguishable from the frequencies of MR signals in the sequence, e.g. by lying outside or on an unused interval of the RF band used by the MR signals. The specific frequencies and the required distance from frequencies occupied by MR signal depends on a number of factors such as the type of pulse sequence run by the MR apparatus, the object in the scanner, the type of non-MR data, the modulation scheme of the non-MR data to the carrier signal, etc.

To ensure that the MR apparatus will transmit the received non-MR data signal undistorted, the carrier signal preferably has a frequency in the range of one or more channels of the MR apparatus.

The non-MR data modulated RF signal may be transmitted in a wire or cable connected to a receiving part of the MR apparatus, or wirelessly to a receiving coil of the MR apparatus. In any case, the electric or EM, Non-MR data modulated RF signal is preferably introduced into the signal path for MR data signals at a point residing inside the magnetic resonance apparatus room.

During periods of a MR recording pulse sequence, the variations in the magnetic gradients in the scanner are so strong that many electrical signals will be affected. At other periods, the magnetic gradients are kept at least substantially constant, and here, handling of other electrical signals in or near the scanner is possible. It can therefore be important to limit the bandwidth of the non-MR data by gating to remove noise in the non-MR signal by band-pass filtering (e.g. thermal). Hence, the method may further comprise the step of gating said electric or EM non-MR data signals in predetermined periods of the MR sequence (sample-hold). Alternatively or supplementary thereto, as the sequence typically applies those ‘silent’ periods for recording MR-data signals, the method may instead comprise the step of synchronising a measuring of said electric or EM non-MR data signals with a measuring of a MR data signal obtained during the MR sequence. Also, after digitalization, the non-MR data is preferably recorded in synchrony with MR data obtained during the same MR sequence. This allows correlation between e.g. the activity determined from an EEG and the corresponding fMRI data.

Typically, the RF modulated non-MR data is received and recorded at the MR apparatus during a MR sequence and together with a MR data signal from a scanned subject or object. Even if the recording sequences of MR and non-MR data does not overlap in time, the invention still presents a huge advantage since the MR apparatus replaces some of the additional apparatuses involved with the non-MR data. In the prior art, each type of non-MR data signal would involve at least a sampler, filters (for getting signals out of the cage). These apparatuses would have to be designed for use inside a MR room or turned off and possibly removed from the room during scanning. According to the present invention, the MR-apparatus samples the non-MR data signals and transmits out of the cage through high quality filters. A lot of the equipment specially designed for MR environments are therefore no longer necessary with the principles of the present invention.

In the field of MR imaging, the scanned subject is often a human or animal in which case it is often relevant to monitor the physiological state of the subject during scanning. Thus, according to a second aspect, the invention provides a method for obtaining a physiological signal of a subject positioned in a magnetic resonance scanning room during a magnetic resonance scanning sequence, the method comprising the steps of:
sensing and/or monitoring a non-magnetic resonance (non-MR) physiological signal of the subject,
modulating the physiological signal onto an electric or electromagnetic carrier signal having a frequency in the range of one or more channels of the magnetic resonance apparatus,
transmitting the modulated carrier signal to a magnetic resonance scanner,
receiving the modulated carrier signal at the magnetic resonance scanner,
digitizing the modulated carrier signal together with MR signals recorded during the sequence, and
demodulating the modulated carrier signal to restore the physiological signal.

The sensed and/or monitored physiological signal may be an electric or electromagnetic physiological signal, or the sensor may be an electric or electromagnetic sensor so that the output physiological signal is an electric or electromagnetic physiological signal. Alternatively, the output physiological signal may be in the form of an optical signal, such as a light signal or a plasmonic signal, in which case the method further comprises the step of converting the sensed and/or monitored physiological signal to an electric or electromagnetic physiological signal. The conversion may be performed before or simultaneous to the RF modulation using an appropriate optoelectronic modulator.

The method may further comprise the steps of amplifying the physiological signal before the RF modulation with an appropriate electric or optic amplifier and transmitting the sensed and/or monitored physiological signal to the RF modulator.

In a third aspect, the invention provides a MR apparatus for implementing the above methods, i.e. a MR apparatus for receiving electric or EM carrier signals modulated with non-MR data during a MR sequence and restoring the non-MR data, the MR apparatus being configured to receive non-MR data modulated carrier signals and transmit the received signals along a signal path of MR signals, the MR apparatus comprising software for accessing received and digitised MR signals and non-MR data modulated carrier signals and, using knowledge of a carrier frequency of the non-MR data modulated signals, identifying non-MR data modulated carrier signals and, using knowledge of the modulation of said non-MR data modulated signals, demodulating identified non-MR data modulated carrier signals and restoring the non-MR data or a function thereof.

Preferably, the MR apparatus further comprises software for synchronising a time stamp on the restored non-MR data with a time stamp on corresponding MR data.

In a fourth aspect, the invention provides the use of a prior art MR apparatus for implementing the above method, i.e. the use of a MR apparatus for receiving, transmitting, digitizing, identifying and demodulating non-MR data modulated carrier signals generated inside a magnetic resonance room during a MR sequence.

Preferably, the MR apparatus is also used to measure the non-MR data modulated signals in synchronisation with predetermined periods of the MR sequence or with measuring of MR data by the MR apparatus.

As the RF signals generated by precessing nuclear spins are very weak, MR apparatuses are very sensitive. All MR apparatus will therefore record almost any non-MR RF signal present as noise (which is of course why the scanner is situated in a Faraday cage). According to the present invention, it is the high sensitivity towards RF signals which is used when non-MR RF signals are controllably generated, and recorded by the MR apparatus.

The methods according to the present invention are novel and inventive over recording of RF noise in the prior art in that the non-MR data signals are deliberately and intentionally modulated to a RF frequency which is within the range of the MR apparatus but outside or in the outskirts of the frequencies of the MR signals of relevance. The non-MR RF signals according to the invention are therefore not equivalent to RF noise or other unwanted signals since they appear in well defined regions in an image or spectrum, and since they are prepared for demodulation for restoration of the non-MR data.

Although other MR apparatuses have been used to record RF noise, the apparatuses according to the present invent are novel and inventive over corresponding apparatuses of the prior art in that it is modified, prepared, adapted or suited for, or comprise means for, or is used in identifying non-MR signals introduced in the MR data signal path, and demodulating the non-MR data signals to restore the non-MR data.

Typically, the present invention will be carried out using a system which is a combination of a modulator for modulating the non-MR data onto signals which can be handled by the MR apparatus, a MR apparatus, and software for performing the identification and demodulation of the non-MR data signals. The system may consist of separate parts or may be build-in and integrated in a MR apparatus. According to a fifth aspect, the invention provides a system for recording non-MR data generated inside a MR measurement room, the system comprising a modulator to be positioned inside the MR measurement room and a MR apparatus, the modulator comprising
a input part for receiving a non-MR data,
a modulating part for modulating the received non-MR data onto a magnetic, electric or electromagnetic signal to form a modulated carrier signal having a frequency in the range of one or more channels of the magnetic resonance apparatus,
   the MR apparatus being adapted to receive modulated carrier signals from the modulator and transmit the received signals along a signal path for MR signals, the MR apparatus comprising software for identifying and demodulating the modulated carrier signals received from the modulator to restore the non-MR data.

The software may be associated with the MR apparatus in that it is to be executed by a computer forming part of the MR apparatus, or in that it is to be executed by a computer to which at least the digitised non-MR data can be transferred from the MR apparatus.

In a preferred embodiment, the modulator is partly integrated with the MR apparatus in that it is integrated with a detection coil for a MR apparatus. Therefore, in a sixth aspect, the invention provides a coil for a MR apparatus, the coil being configured to receive and introduce electromagnetic, radio frequency (RF) MR signals and electric non-MR data signals into a signal path of a MR apparatus, the coil comprising

a receiving coil part for receiving electromagnetic RF MR signals, generating corresponding electric RF MR signals,
a socket part for receiving electric non-MR data signals,
a modulator electronically connected to the socket part, the modulator comprising
an oscillator for generating an electric carrier signal having a frequency in the range of one or more channels of the magnetic resonance apparatus, and
a modulating part for modulating received electric non-MR data signals to the carrier signal,
   one or more electric connections from the receiving coil part and the modulator to the MR apparatus for introducing the electric RF MR signals and the modulated carrier signals into the signal path of the MR apparatus.

Modulators for modulating an incoming signal onto an RF carrier signal exist in the prior art. The application of such a modulator to modulate incoming data to a RF signal which is transmitted to and recorded by a MR apparatus has not been disclosed in the prior art. Hence, according to a seventh aspect, the invention provides the use of a modulator for receiving and modulating non-magnetic resonance (non-MR) data generated inside the scanner room onto a magnetic, electric or electromagnetic signal to form a modulated carrier signal and transmitting the modulated carrier signal to a magnetic resonance apparatus, the modulator being positioned inside a magnetic resonance apparatus room and operating during a magnetic resonance sequence, the modulator comprising
an input part for receiving non-MR data,
a modulating part for modulating the received non-MR data onto a magnetic, electric or electromagnetic signal to form a modulated carrier signal having a frequency in the range of one or more channels of the magnetic resonance apparatus, and
a transmitting part for transmitting the modulated carrier signal to the magnetic resonance apparatus.

The present invention provides a new and inventive solution to the problems of image and signal artifacts, synchronization, data management and bandwidth presented in relation to the prior art. The invention provides a particularly simple method for recording non-MR data signals. In a preferred embodiment, the non-MR data or data signals is modulated onto RF signals which can be received by the MR apparatus together with the normal MR signals. Here, the RF carriers are emitted from a frequency generator and are modulated by electrical signals that are to be measured during imaging. In an alternative embodiment, the modulated carrier signals are fed to the MR apparatus at a later stage in the signal path of the MR apparatus.

These modulated RF signals emitted within the RF enclosure are recorded by the MR apparatus and can subsequently be extracted from the MR images or spectra. The modulated signals can be emitted as radio waves inside the RF enclosure by use of a simple aerial, thereby ensuring galvanic separation between the subject and the recording apparatus. Exploiting surplus bandwidth, the MR system is therefore used for recording and storing both MRI and electrical signals with inherent microsecond synchronization.

In order to limit the bandwidth of the non-MR signal and/or avoid the gradient and RF induced noise from causing transients in the measured EPH-signals, gradient activity triggered gating can be implemented with a sensor coil placed near the opening of the scanner.

In addition to the MR equipment, only relatively simple, light, safe and low-cost equipment is needed. If raw data is available from the scanner, sequence modification is normally not needed. The method is highly generic and is compatible with most existing MR systems with no need for hardware adaptation or interfacing.

An added, important advantage is that the technique offers an implementation of EEG-recording techniques presented by Anami et al (Neuroimage 19, 281) and Cohen et al. (Proc. of Human Brain Mapping Annual Meeting. 2001; 6). It is therefore considered highly relevant for EEG-fMRI.

In the instrument visualization described previously, self-resonant RF circuits are attached to instruments to act as high-contrast markers for localisation purposes in MR imaging (MRI), see e.g. Weiss et al., Proc. 9th Annual Meeting on ISMRM, 2001, p. 544 or Wong et al., J magn. reson. Imaging 12 (2000), p. 632. Here, a RF coil is tuned to the Larmor frequency of the scanner modifies the effective excitation angle for the substance inside the coil, whereby the interior of the coils can be brightly depicted. In order to turn the visualisation on/off, the RF circuit can be tuned/detuned by an optical signal from the scanner using a photodiode connected to the RF circuit. Turning something on/off is only a modulation if it forms part of an encoding scheme where a series of on/off's are provided to convey additional data (e.g. in the form of binary data). This is clearly not the object in the instrument visualization applications. Hence, no data is modulated onto the MR signal from the interior of the coil and as a consequence, there is no demodulation of data or data signals from the acquired image.

The applications of quartz crystals as temperature sensors described previously make use of inherent-material responses to the fields from the MR apparatus. It does not present a deliberate modulation of a signal onto a carrier, instead, the carrier is consequently and inevitably formed with this variation. This is the same way as the resonance frequencies and relaxation times of nuclear species depends on its molecular environment, e.g. oxygenated or deoxygenated hemoglobin.

FIG. 1 of Simon (J. magn. reson. 128 (1997), p. 194) shows signals received by the MR scanner from a quartz crystal. The signals provide the temperature increase of the crystal (expressed as a drift in the resonance frequency) over time. Although the temperature increase is caused by a signal or energy received by the crystal, the recorded signal cannot simply be demodulated to give the temporal evolution of the signal or energy causing the temperature increase. The person skilled in the art will observe that the signals in this figure clearly shows a gradual slowing down of the temperature increase as the temperature approaches its maximum. This is a natural consequence of the thermodynamics of heat transfer where the rate of heat transfer is proportional to the temperature difference. Thus, FIG. 1 of Simon shows the heating curve resulting from turning on the heat source for the quartz crystal. Thus, the temperature sensors of Wang & Leigh and Simon does not disclose modulation of a signal received by the crystal to the RF signal from the crystal, nor transfer of such signal to the MR apparatus with subsequent demodulation.

### BRIEF DESCRIPTION OF THE FIGURES

In the following, the invention and selected embodiments thereof will be described in relation to the accompanying figures in which:

FIGS. 1 and 2 illustrate preferred embodiments of the overall layout of the present invention.

FIG. 3 illustrates the configuration of a modulator for use in and according to the invention.

FIG. 4 illustrates a signal path for MR data signals in a MR apparatus.

FIG. 5 shows a coil having a modulator according to an embodiment of the invention.

FIG. 6 shows a preferred embodiment for recording of electrophysiological signals from a patient during scanning.

FIGS. 7A and B are diagrams of typical Echo planar imaging pulse sequences.

FIG. 8 shows a circuit for use as a modulator according to the invention.

FIG. 9 is a flow diagram illustrating software for extracting a non-MR data signal from data recorded by the MR apparatus.

FIG. 10 shows the configuration for an amplifier used to amplify EPH signals to be used in a preferred embodiment of the invention.

FIG. 11 shows simultaneous acquisition of an EPI image as well as three non-MR signals (ECG, EOG and calibration) transmitted wirelessly to the scanner. The non-MR signals are seen as vertical patterns in the image.

FIG. 12 shows spectrograms calculated from all the EPI line readouts for repeated recording of images similar to FIG. 11.

FIG. 13 are graphs showing the time courses of the encoded non-MR signals extracted from MRI raw image data.

FIG. 14 shows a matrix of cross-correlation maps between the measured time courses of FIG. 13 and the acquired series of MRI images.

FIG. 15 is a Lomb-Scargle periodogram calculated from extracted data similar to the graphs of FIG. 13.

### DETAILED DESCRIPTION

In a preferred embodiment, the present invention provides a method and a system for recording EPH signals, subject responses and other signals to be generated inside a MR room and recorded during a MR scanning or spectroscopy. In other aspects, the invention relates to an MR apparatus or a receiving coil of an MR apparatus configured to perform such recording. In yet other aspects, the invention relates to the use of a MR apparatus or a modulator for making such recordings. These aspects will all be explained in the following in relation to a number of preferred embodiments.

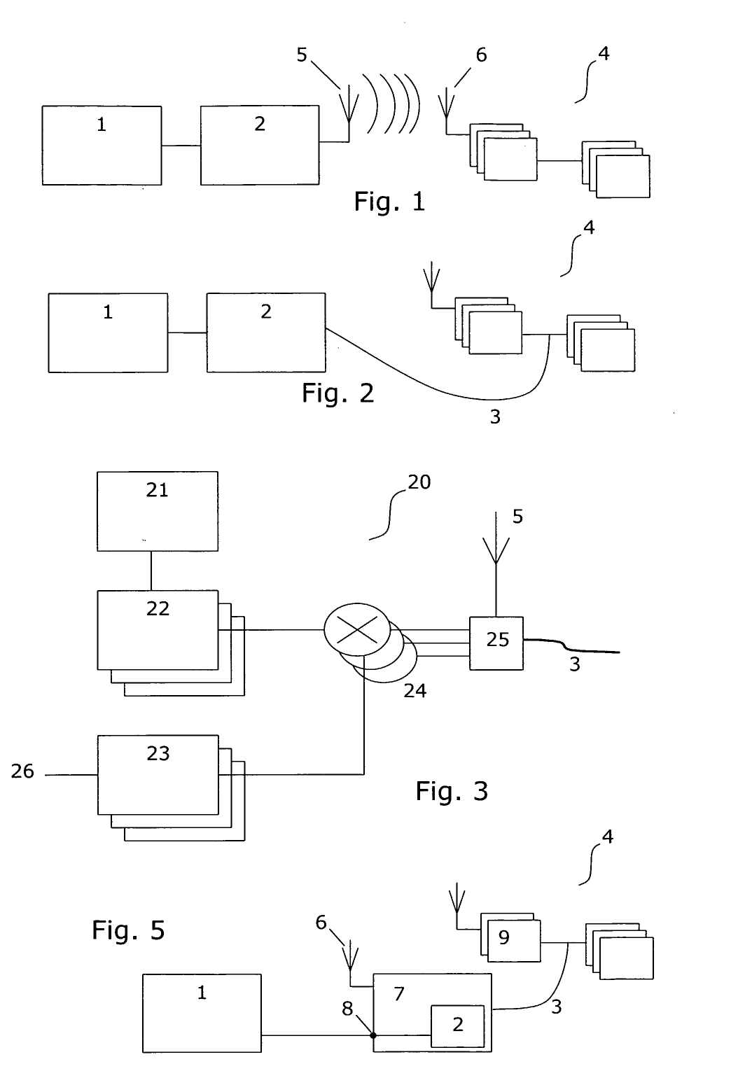

In a first preferred embodiment, the method for recording non-MR signals according to the invention can involve a number of distinct steps described in the following with reference to FIGS. 1 and 2. Depending on the specific use, not all steps need to be present, and some steps may be performed simultaneously.

Steps I and II take place in a pre-processing unit 1 for detecting and processing non-MR signals.

### Step I

Analogue or digital non-MR signals are generated by an output from a signal source. Examples:
EPH signals generated from animal or human body.
Signals generated by a measurement apparatus, e.g. an electric or optical sensor.
Subject motion converted to electric signals by means of, e.g., computer mouse, joystick, or pushbuttons.
Output from a signal generator, such as a clock.

### Step II

The non-MR signals may be prepared for further processing, e.g., by any combination of amplification, conversion, splitting, combining, digitization, mixing, gating, delaying, filtering etc. The process possibly involves a conversion of the signal to an electric signal. Examples:
EPH signals are amplified, offset and fed to a sample-hold circuit, triggered by another signal such as that generated by a detector of magnetic field changes.
A signal may be split into parts that are buffered and delayed differently.
An optical signal is converted to an electric in an opto-electronic converter, e.g. a photodiode.

### Step III

Step III takes place in a modulator 2, the non-MR data signals are encoded onto one or more electric or electromagnetic carrier signals with a frequency which can be handled by the relevant part of a MR apparatus 4. The one or more modulated electric or EM RF carrier signals are transmitted to the MR apparatus by a wired connection (electrical) or wirelessly (electromagnetic). Examples:
The non-MR data signals are encoded by amplitude modulation onto individual carrier signals at separate RF frequencies. The RF signals are combined and transmitted to the scanner 4 as radio waves by use of a transmitting aerial 5. The non-MR signals are received by the inherent receiver coil 6 of the MR apparatus 4 during a normal MR spectroscopy or imaging sequence.
The non-MR data signals are encoded by amplitude modulation onto individual carrier signals at appropriate frequencies. The signals are combined and transmitted by wire or cable 3 to a combiner somewhere along the MR data signal path of MR apparatus 4, thereby inserting the non-MR data signals in the MR data signal path.
The non-MR data signals are used to modulate the magnetic field locally in the MR receiver coil so that the individual signals are encoded in the Larmor frequencies or amplitude decay times of radio waves emitted from substances exhibiting magnetic resonance.

### Step IV

The non-MR data signals are introduced somewhere along the normal scanner/spectrometer signal line, and are possibly digitized. They may, for example, be measured during scanning using a normal MR spectroscopy or imaging sequence and receiver coil, so that the non-MR signals are embedded in the (complex) MR raw data or images. Alternatively, if a relatively low frequency (e.g., 100 kHz) is chosen in step III, the signal can be mixed into the MR signal path at a low frequency stage immediately prior to digitization.

As the non-MR signals are introduced in the MR data signal path, they are processed similarly to the MR data signals and thereby possibly embedded in the MR raw data or images.

### Step V

The original non-MR signals generated in Step I, or derived functions of these, are extracted from the MR raw data after digitization. Scaled and offset sampled intervals of the original non-MR signals may, for example, be derived from imaging raw data.

The method for recording non-MR signals outlined above can be implemented by existing MR apparatuses or by new MR apparatus which have been designed to implement this method.

### Step VI

The images and electrical signals are stored, e.g. together in a radiological picture archiving system (PACS) where the scanner may send the images anyway. The DICOM data format (Digital Imaging and Communications in Medicine) used by most PACS implementations supports both EPH- and MR-image formats.

In a first embodiment, the above method is implemented by existing MR apparatus, an amplifier with few modifications as well as one specially designed modulator. In the following, the equipment applied in the first embodiment is described in detail.

The encoding of the non-MR signal outlined in Step III above can be performed using a specially designed Multi frequency amplitude modulator (MFAM) 2 in FIGS. 1 and 2. In a preferred embodiment, the modulator 2 is a MFAM 20 shown in FIG. 3, comprising a common reference oscillator 21 and eight oscillators 22 providing different frequency offsets for each of a total of eight channels.

The reference oscillator 21 is a very stable and programmable oscillator made with a voltage controlled crystal oscillator (VCXO) and a low jitter programmable phase locked loop (PLL). This gives the advantage of easy adjustment to the MR frequency range. The clock frequency for each oscillator 22 is obtained by individual low jitter programmable PLLs. The digital output from each PLL is bandpass filtered to avoid problems from higher harmonics. The frequencies of operation are chosen within the bandwidth of the MR apparatus and its components i.e. filters, but outside the frequency range of important MR data signals.

The non-MR input signals of Step I and II are received at inputs 26 and are low-pass filtered by filter 23 to avoid channel interference and distortion of the MR signals. The channel clocks and the filtered signals are fed to amplitude modulators 24. The outputs from all the amplitude modulators are joined in a power combiner 25 and the RF output is sent to the MR apparatus either cable 3 or transmitting aerial 5.

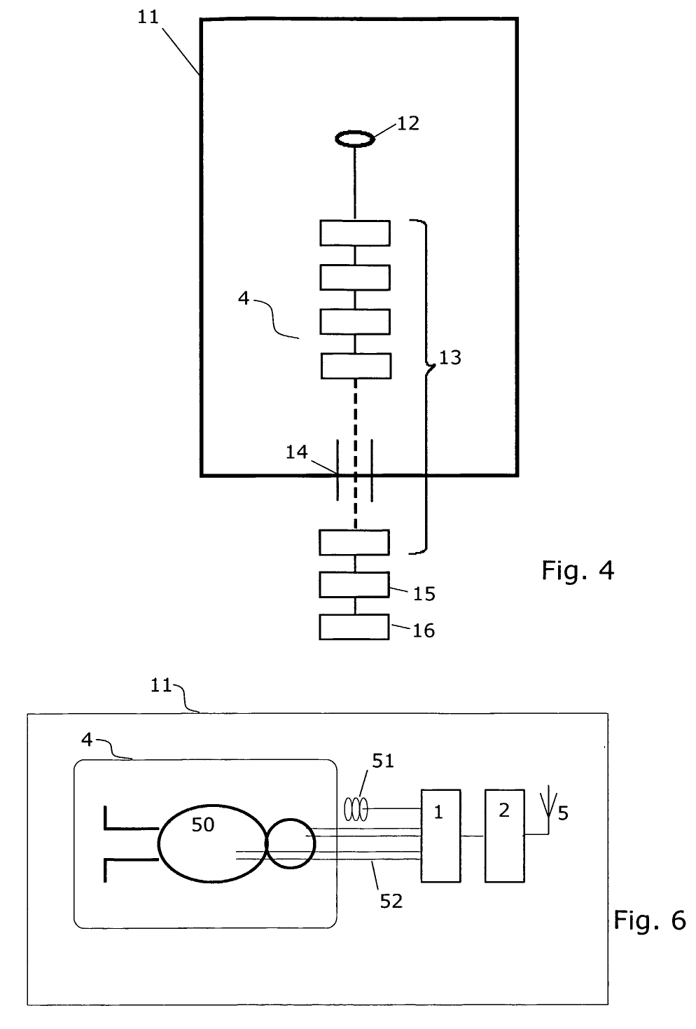

FIG. 4 illustrates the normal signal path of a MR apparatus 4 with one coil element and one receiver channel. When more channels are present, principally similar parallel signal paths may be present. The signal paths may join in common components.

Normally, the measured RF signal from the precessing protons is detected in one or more RF coils 12 of the scanner. The signals from separate coil elements are either combined and processed together, or are processed separately in more or less independent RF channels that may operate at different frequencies. The MR data signals are amplified and modulated down to a relatively low frequency suitable for digitization. This amplification and modulation happens in one or more steps along part 13 of the signal path which may reside both inside and outside of the faraday cage 11. At the boundary of the faraday cage 11, a penetration panel 14 prevents irrelevant RF signals from the outside to be transferred to the faraday cage and detected by the RF coils. As no new signal paths are introduced to transfer the non-MR signal of interest out of the faraday cage 11, the invention involves no alterations to the penetration panel. Before storage in memory 16, all signals in the signal path, i.e. both MR and non-MR if present, are digitised in analogue to digital converter (ADC) 15. Eventually, the stored MR data signals from the separate channels are often combined to form images or spectra.

The non-MR signals can be introduced into the MR data signal path at several stages. In the wireless embodiment using transmitting aerial 5 of FIG. 3, the signals are introduced via a normal or dedicated RF-coil tuned to the frequency of the modulated non-MR signal as determined by the MFAM (which may be within the MR-frequency range).

It is equally possible to introduce the signal at later stages of amplification or modulation in the signal path, e.g. directly into an unused coil input port via a galvanic isolation. In principle, the signal can be introduced at any stage of the signal path within cage 11 at a frequency giving detectable changes in the digitised signal, e.g., at a low frequency immediately before digitization. Here, a cable 3 between the RF output of the MFAM and the MR apparatus will most typically be used, although it would also be possible to apply the wireless solution using dedicated coil and circuitry. As the detected MR signal is modulated from the Larmor frequency to lower frequencies along the signal path, the modulation frequency of the non-MR data signal should be adjusted accordingly depending on the point at which it is introduced.

The non-MR signal can be introduced in channels that carries also MR-signal, or it can be in separate channels. The latter option having the advantage that separation of MR and non-MR signals is inherent. As an example, several non-MR signals may be multiplexed or in other ways mixed, before being introduced via a transformer into a channel presently not used for MR-signal (even though usable for MR-signals also).

The electric connection between cable 3 and the MR data signal path is made through a transformer with galvanic isolation.

Being introduced into the MR data signal path, the non-MR data signals are processed equivalently to the MR data signals in the remaining signal path. This processing include analogue to digital conversion in ADC 15 and storage in memory 16. Analysing tools provided in relation to or implemented in the MR apparatus can be used on the raw MR data or images to demodulate and restore the non-MR signals in connection with the corresponding MR data.

In a second embodiment the functions performed in Steps III and IV are reversed and incorporated in the MR apparatus. Here, the MR apparatus comprises means for registering, measuring or monitoring non-MR signals in the scanner room during a sequence and for modulating and transmitting the non-MR signals along the MR data signal path. As for the first embodiment, analysing tools connected to or implemented in the MR apparatus can demodulate and restore the non-MR signals in connection with the corresponding MR data.

An attractive implementation of the means for registering, measuring or monitoring non-MR signals in the scanner room involve the equipment of an otherwise normal coil with one or more dedicated connections for external signal leads, e.g., sockets for EEG electrodes. The MFAM and galvanic separation from patient could be implemented in the coil, and the data would be transferred through a preferably (but not necessarily) unused channel of the scanner. Also, the input signals could be amplified or the amplifier could be implemented in the coil as well. With a special sequence treating signals from channels differently (when separate channels are used), or splitting MR- and non-MR data signals, the non-MR and MR signals could be reconstructed separately.

In another embodiment, the means comprises input ports to a combiner incorporated along the normal MR data signal path as well as MFAM and galvanic separation. An additional non-MR apparatus in the cage could be connected directly to these input ports. Here, the oscillators of the MFAM should be tuned to provide modulation frequencies suitable for the relevant stage of the signal path. Similarly, if the means includes an amplifier, the amplified signals should correspond to the equivalent MR-data signals at that stage. The non-MR signal can thereby be introduced at other positions along the paths, which may be preferable, e.g. for avoiding potentially noise-emitting equipment operating at the Larmor frequency.

Thus, using the proposed method and with minor modifications of existing coil and sequence technology, non-MR signals of interest can be measured with a normal MR apparatus during in an otherwise normal MR measuring sequence.

The modulator or MFAM can be fully or partly incorporated into the MR apparatus. In this embodiment, shown in FIG. 5, a coil 7 of the MR apparatus has a receiving coil part or antenna 6, a modulator 2, and an input terminal 8 for receiving a non-MR signal from a pre-processing unit 1. The pre-processing unit 1 and modulator 2 have been described in the above. By incorporating the modulator in the coil 7, the non-MR data signal can be fed directly into the MR apparatus whereby the amount of wires and apparatuses in or near the coil can be reduced. From the coil, the modulated carrier signal is introduced in the signal path of the MR apparatus 4 together with the normal MR signal picked up by the receiving coil 6.

In the case of advanced MR-imaging it is often of interest to monitor physiological parameters of a subject during scanning. These include:
1. Eletrophysiological parameters reflecting the status of the subject as reflected in electric fields of the human body including electrocardiograms (ECG), electromyograms (EMG), electrooculograms (EOG) and electroencephalograms (EEG).
2. Other measures of subject condition including pulse, respiration (e.g. measured by pneumatic belt), blood saturation (e.g. measured by pulseoximetry), blood pressure, temperature (e.g. measured by thermocouple), expiration gas concentrations (e.g. measured with capnograph). Some of these signals are pneumatic or optical in their nature but are easily converted into electrical signals.

Here, the non-MR data signal is a physiological signal for monitoring the state of the subject. It is a well recognised problem that the violent changes in magnetic gradients produced by the scanner introduce signal artifacts in the physiological measurement which can normally only be overcome by precise synchronisation or retrospective filtering.

According to the state of the art, apparatus for recording physiological signals as mentioned above typically consist of a set of sensors and dedicated apparatus for amplification, sampling, analogue to digital (AD) conversion and storing. The sampling must be performed in precise synchronisation with the scanning sequence of the MR apparatus to avoid large signal artifacts in the physiological signal. Also, the sampler etc. must be MR compatible in the sense that they can tolerate the electromagnetic fields associated with MR-scanning and in that they are RF shielded in order not to generate unwanted electromagnetic fields at MR-frequencies. When the physiological signal is to be used outside the scanner room, careful filtering has to be made in order not to disturb the shielding properties of the Faraday cabin in which the scanner is located.

According to the present invention, there is no need for dedicated sampling and AD converting apparatuses when recording physiological signals. FIG. 6 illustrates a preferred embodiment for recording of EPH signals inside the scanner room 11 according to the invention. Here, amplifier 1 amplifies the electrophysiological signals from e.g., eye and heart musculature of patient 50 as described previously. As is customary in MR compatible EPH equipment, twisted electrode pairs 52 are used. To limit the bandwidth of the non-MR signal, a gradient activity (GA) trigger circuit 51 is used to trigger the sample-hold circuit. The amplified signal is used by MFAM 2 to modulate the amplitude of carrier signals at distinct frequencies in the detection range of the scanner. The signals are emitted in the scanner room by a simple aerial 5 and are recorded by the scanner 4 along with the normal MR data signal. As the electric physiological signal is inserted into the signal path of the MR data signals, the MR apparatus acts as the sampling, filtering and AD converting apparatuses for the non-MR signals. This also solves the problem of getting the physiological signals out of the scanner room, rendering the dedicated apparatus superfluous. The sampling part of the MR apparatus provides inherent microsecond synchronisation with the scanning sequence so that gradient artifacts in the physiological signals can be essentially avoided.

Due to the large bandwidth of the MR apparatus, it can handle a large variety of physiological signals. Each piece of equipment for measuring a physiological signal could be equipped with its own MFAM operating at different frequencies, or they could be wired to a common MFAM which would assure that no two signals are transmitted with the same carrier frequency. Using several channels from a multi-channel MR-scanner the method could even be useful in extreme cases, such as getting the signal from hundreds of EEG-channels out of the scanner room.

The equipment feeding the physiological signals to the MFAM (e.g. electrodes and cables and possibly an amplifier) should be MR compatible in the sense that they can tolerate the electromagnetic fields associated with MR-scanning and do not generate unwanted electromagnetic fields at MR-frequencies themselves. This would typically include RF shielding of circuitry and avoidance of ferromagnetic materials in electrodes and other parts to be situated within the scanner.

Recording MR images involves a large variety of different pulse sequences which determine the types of tissue being imaged, contrast etc. Commonly used pulse sequences are Spin echo sequence, Inversion recovery sequence, Gradient echo sequences, Echo-planar pulse sequence and more. Practically, a pulse sequence consists of many short periods in succession where the MR apparatus generates different EM field conditions (magnetic and RF fields) inside the scanning part. At periods with large changes in the magnetic gradients, the signal artifact on the non-MR data signal will be very large. Therefore, it is desirable to synchronise the sampling of the non-MR data signals by the MR apparatus with periods where the magnetic gradients are constant or slowly varying.

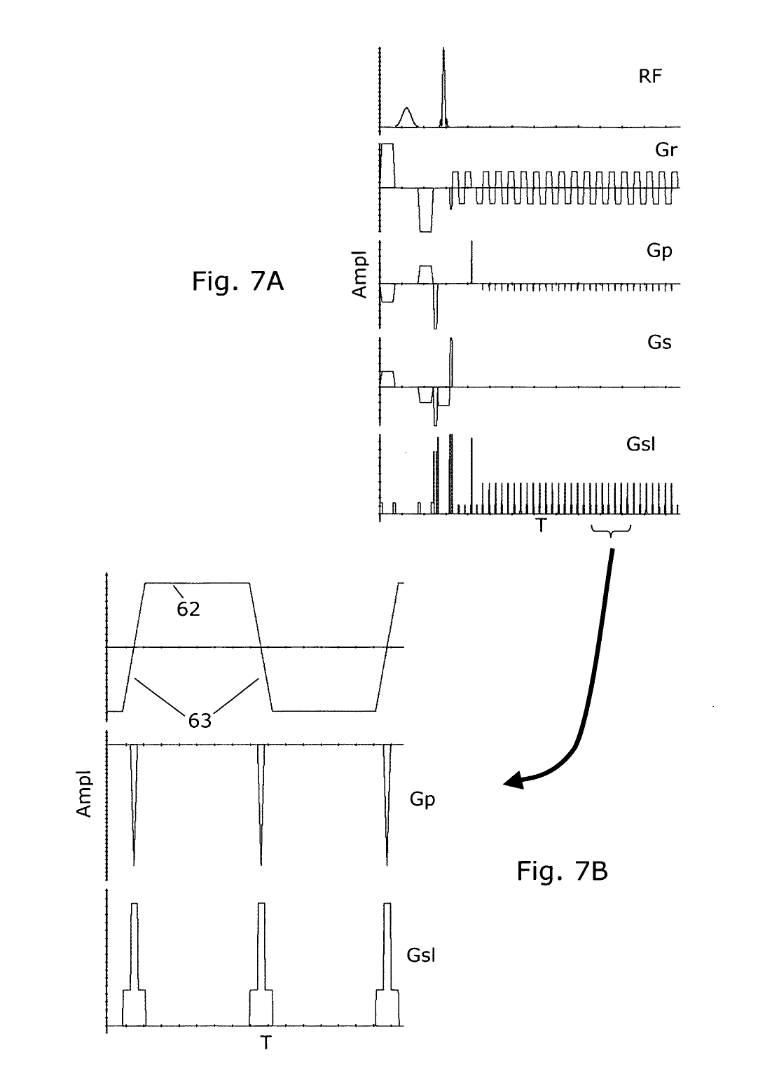

FIG. 7A shows a schematic diagram of RF emissions and gradient pulses in a typical functional MR imaging (fMRI) sequence (Anami et al. Neuroimage 19, 281). The following denotations applies:

| | |
|---|---|
| RF, radio frequency wave | Gs, slice selection gradient, Z-gradient. |
| Gp, phase encoding gradient, | Gr, readout gradient, X-gradient. |
| Y-gradient. | |
| GsI, Gradient slew rate | |

The gradient slew rate, Gsl, is the numerical value of the time-derivative of the strength of the magnetic gradient, summed over the X, Y and Z gradients. The gradient slew rate is proportional to the expected artifact or noise in a recorded EEG signal. The artifact corresponding to each gradient component can be identified. Using a stepping stone readout gradient Gr as shown in FIG. 7B, the readout gradients are not sinusoidal but have plateaus 62 of constant magnetic field gradients of e.g. 800 μs between up/down ramps 63 of e.g. 200 μs. The plateaus 62 allow for short artifact-free periods in the EEG record where sampling can be performed.

This technique requires microsecond-synchronisation between the fMRI sequence and the EEG sampling which can be difficult to achieve. With the MFAM, overlap between gradient switching and EEG sampling can easily be avoided by modifying the MR-sequence to only sample the MR and non-MR signal when the magnetic gradients are of constant amplitude. Most standard pulse sequence implementations will only require changes at a post-processing level to extract the non-MR signal.

After AD conversion of the combined MR and non-MR data, the raw data are stored on a hard disk or equivalent storage media. If the operator can gain access to the raw data, it is possible to design software which can extract and restore the non-MR data, provided that the measurement timing, the oscillation frequencies and the modulation scheme from the modulator (MFAM) is known. For slowly varying non-MR data, such as monitoring of breathing, it may not be necessary to access raw data to extract the non-MR signal.

Echo planar imaging (EPI) sequences are the most commonly used for functional MRI. In the following, the decoding of MR-and non-MR signals acquired simultaneously with an EPI sequence is described for the case where non-MR signals are encoded in the RF-carrier by amplitude modulation. The basic principle for extracting the measured non-MR signal is to ignore the imaging gradients as these do not influence the measured non-MR signal directly (except possibly for gradient induced artifacts). With this in mind, extraction of the non-MR signal is simple, even when gradient patters are complicated. The general principle can therefore be used for signals acquired with other imaging sequences too.

Images acquired while the MFAM is active in the scanner room appear normal except for patterns along lines orthogonal to the frequency encoding (readout) direction. Depending on the chosen modulation frequencies of the non-MR data signals, these lines may only be visible when the total sampling bandwidth is increased. With appropriate adjustment, the image of the scanned subject is undistorted. Hence, the MFAM produce very controlled image artifacts or noise signals containing reproducible data, and this artifact or noise lies nicely along the edge of the produced images without disturbing the object of the image.

Image acquisition using EPI involves high bandwidth sampling, typically around 100 kHz for approximately 100 ms. Often, the sampling period is split into shorter periods, one per line of the image. In contrast to the MR signals, the non-MR data signals emitted from the MFAM are not directly influenced by changing magnetic fields. With the chosen encoding (amplitude modulation at distinct frequencies), the encoded signals can consequently be extracted from columns of a spectrogram of the measured data. It is simple and fast to calculate a spectrogram for each image, i.e., the frequency content as a function of time. This is most easily done by organising the measured data in a matrix similar to the k-space data matrix used for image reconstruction, but with time along both axis (“true” time, and time from last beginning of a sample period). For the case of EPI, this matrix is identical to the k-space data matrix except for a reversal of every second line, and possibly an apodization. In order to calculate the spectrogram, the matrix can be Fourier transformed along the “short” time axis.

It was assumed implicitly above that the original signal and therefore the amplitude of the RF signal, does not change faster than the line time (typically 500-2000 us). This, however, is not a serious constraint, as the spectrogram can also be calculated from other groupings of the data along the two time-axis, thus allowing a trade-off of time-resolution for spectral resolution.

The squares of encoded signals can be reconstructed with, e.g., 1 ms time resolution by weighted averaging of a small number of neighbouring columns of the squared absolute spectrogram.

The outlined principles are equally valid for other imaging sequences, multi-slice imaging and imaging over longer periods of time. The encoded signals can therefore be measured simultaneously with normal imaging using the scanner. Extra sampling periods can be inserted in waiting periods to increase sampling density. Another way to increase the sampling density is to use the method outlined in example 2 in step II above: The time-displaced signals can be recombined to increase sampling density.

Since longer periods of the physiological data are acquired over many images, there are short periods during spin preparation, where no physiological signals are measured. Consequently, the frequency content needs to be estimated from uneven sampling, e.g., using gridding, Lomb-Scargle periodograms or Bayesian estimation.

Another embodiment of the invention relates to the measurement of other imaging modalities than MRI within an MR apparatus. As an example, avalanche photo diodes can be used as detectors in positron emission tomography (PET) scanning and they have been demonstrated for simultaneous PET and MRI. A practical way to integrate the PET detectors in an MR acquisition and post-processing environment is via the technique proposed in the present invention: Signals preferably derived from the coincidence detectors of a PET detector unit can be pre-processed and encoded onto one or more RF carrier signals transferred to the MR apparatus as described. This facilitates the implementation of new imaging modalities, as existing signal paths and available post-processing and visualisation tools already available in the MR environment can be employed.

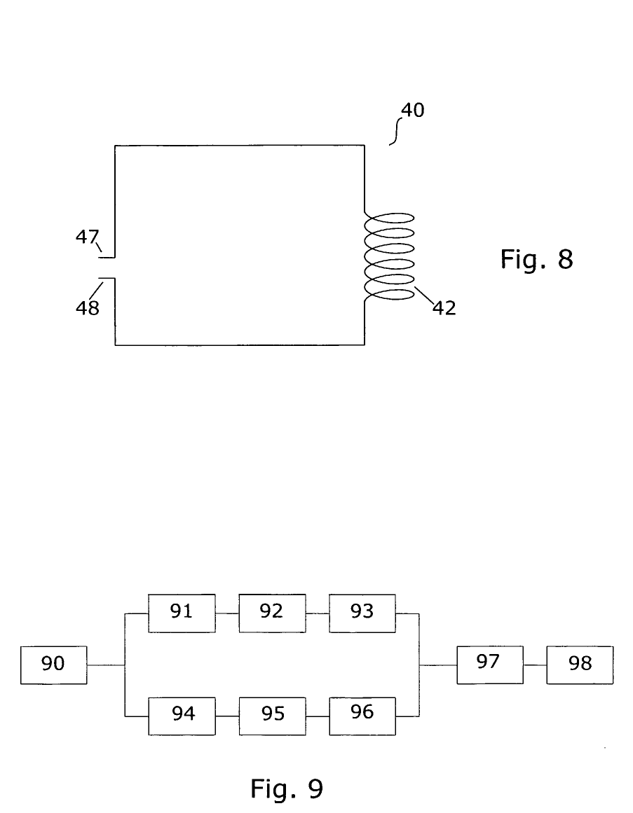

In a further embodiment, the RF carrier is provided by a substance exhibiting magnetic resonance in the scanner. The non-MR data or data signal is modulated onto the magnetic field from the MR apparatus within a delimited region, where the induced MR signal is modulated and used as a carrier. This allows for very small and simple alternatives to the MFAM described in relation to FIG. 3. FIG. 8 shows an embodiment of a simple, miniaturized modulator according to the further embodiment of the invention. The modulator is a circuit 40 with a coil 42 and two input terminals 47 and 48.

When the input terminals 47 and 48 receive an electrical signal from a variable source of electromotive force (EMF, e.g. a battery connected in series to a resistor sensitive to temperature), a corresponding current is generated in the circuit 40 including coil 42, thereby producing a magnetic field inside the coil according to Ampere's Law. The circuit 40 will thereby create a magnetic field modulated with the received electrical signal. When positioned in an MR apparatus, nuclei inside the coil 42 will experience a superposition of the magnetic fields from the MR apparatus and the modulated magnetic field from the circuit.

With proper tuning of the inductance of coil 42, the modulated MR signal from nuclei inside the coil can be extracted from the other MR data from the sequence, and can be analysed to provide the encoded non-MR data.

The modulator of FIG. 8 may for example provide a very simple and easy way of recording subject responses. A few simple contacts designed to give different currents in the coil 42 upon activation can be connected in series with a battery to the input terminals 47 and 48. In one very simple example, two contacts (e.g. a Yes and a No contact) will each shortcut a circuit with different resistance and thus EMF. Depending on the response of the subject, the MR signal will be amplitude modulated with either large or small amplitude and the response from the subject can be decoded from the raw MR data. As the circuit 40 can be made very small, it may also be used to probe the local environment in areas that are difficult to access, e.g. intra-abdominally.

Software for extracting and restoring the non-MR data signals from the data recorded by the MR apparatus has been mentioned. The following describes a scenario for software processing of the recorded data and, given the oscillation frequencies and the modulation scheme from the modulator (MFAM), extracting the non-MR signal.

FIG. 9 illustrates the data flow during post-processing of the MR and non-MR data, the references numbers refer to the following acts:
90: Acquire raw data
91: Limit bandwidth if oversampling is employed
92: Perform normal image reconstruction
93: Store images
94: Calculate spectrogram
95: Determine carrier frequencies
96: Extract and store non-MR signals
97: Correlate MR and non-MR signals
98: Store correlation maps

The data flow is from left to right in the figure. Starting at 90, the measured raw data contains both MR and non-MR components. The data are split for independent extraction of these components. The upper branch illustrates normal MR image reconstruction and data storage, 92 and 93. Special attention should be given to the first step 91 of limiting the raw data bandwidth: If the MFAM frequencies are chosen outside the MR frequency range, this filtering process allows the rest of the MR image reconstruction 92 to be entirely normal and to provide images free of artifacts (i.e. independent of the non-MR signals). Such bandwidth reduction corresponding to oversampling is standard signal processing employed as default for several commercial MR apparatuses.

The lower post-processing branch illustrates how extraction of the non-MR data can be performed when the MFAM frequencies have been chosen outside the frequency range of MR signals. First, 94, a spectrogram of the measured data is calculated. Based on the peaked nature of the power profile from which the MFAM frequencies can be derived, or based on priori knowledge of these frequencies, the non-MR signals can be extracted from the spectrogram and subsequently be stored (95 and 96), e.g. together with the imaging data in a PACS.

Finally, the (now separate) MR and non-MR data will often be brought together again for a correlation analysis (97 and 98) as illustrated in the right part of FIG. 9, e.g., to map the source of epileptic spike activity, as EEG can answer the “when”-question, and fMRI can answer the corresponding “where”-question. The correlation maps can conveniently be presented to the scanner operator or be stored, e.g. in a PACS.

A demonstration setup for measuring several EPH- and calibration signals simultaneously during echo planar MR imaging (EPI) will be presented in the following.

For this ultra-fast imaging method, the signal can be acquired during short periods with no gradient activity. In the demonstration, EPH signals have been measured with electrodes positioned near the heart, eye musculature and brain to record electrocardiogram (ECG), electrooculogram (EOG) and electroencephalogram (EEG) simultaneously. The setup is thus similar to the setup shown in FIG. 6.

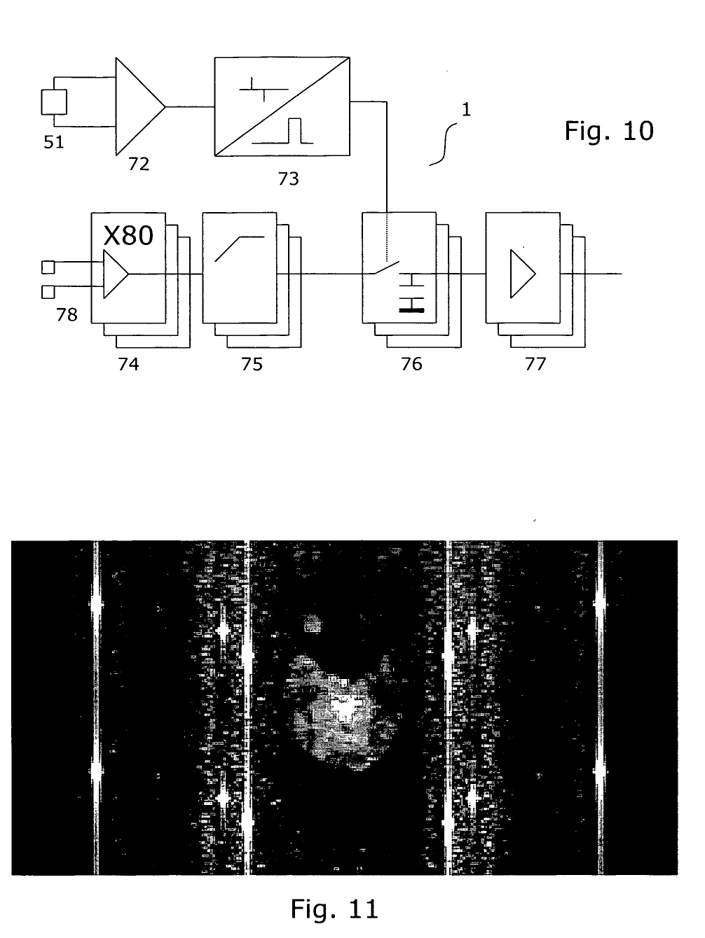

FIG. 10 shows an amplifier 1 for EPH-signals with selective detection (gating) and short settling time constructed to avoid influence from RF pulses and magnetic field changes that could saturate the circuit. As described in relation to FIGS. 7A and B, the magnetic gradients change before each sampling period during echo planar MR imaging. This introduces a short but very powerful unwanted signal decades stronger than the EPH signal. Each sampling period in echo planar imaging is short, typical around 500-2000 us per line, separated by shorter periods of gradient activity. To avoid measuring signals induced by gradient activity, the EPH- and MR signals were sampled and held when transients from the magnetic gradient change had died out. The signals can be delayed for the time of an image acquisition, typical around 100 ms. The measured data may then be captured in a silent condition. Alternatively, the EPH signal can be sampled when the artifact from the magnetic gradient change has died out. This can be obtained with amplifiers with very short settling times and high bandwidth.

The EPH signals was measured with ECG/EEG electrodes 78 and fed to an ECG pre-amplifier 74 with high bandwidth and small settling time. The DC components from the electrodes 78 are removed from the pre-amplified signals by a high pass filter 75, and the filtered signal is fed to the gradient triggered sample hold circuit 76.

Special care is important to avoid saturation of the circuits of amplifier 1. To synchronise the sample-hold circuit 76 with the magnetic field changes, a detection coil 51 was placed within the scanner part of the MR apparatus. The induced signal was amplified by gradient activity detector amplifier 72 and monitored by gradient detection and delay circuit 73 to detect gradient activity. A high filter bandwidth of 1 MHz for the gating can ensure that also abrupt gradient changes are detected. After approximately 100 μs of silence following periods of activity (sufficient for the electrode signal to settle and to avoid the artifact from the RF pulses), the sample hold circuit 76 was opened for around 20 μs. A 10 kHz low-pass filter is associated with this, and the noise level therefore corresponds to 100 μs signal averaging per sample of the undistorted signals. The filter leaves signal at frequencies below 625 Hz essentially undistorted. In total, the delay from gradient activity to sampling was approximately 120 μs which is shorter than the period between gradient reversals even for fast EPI with an echo spacing of say 500 μs. The exact timing is not critical, as long as the amplifier has time to settle. Consequently sampling with practically any plateau-sampling EPI sequence is feasible with no adjustment of the sequence or constructed hardware.

Each channel of the EPH signals were amplified by an adjustable factor (×1600-14600 allowing input ranges of 300 μV to 3 mV, full scale) by amplifiers 74 and 77. Also, the signal was offset and scaled to ensure an always positive sign for the dynamic range of interest. This resolves sign ambiguity in subsequent demodulation steps. Artificially generated step-function signals was generated simultaneously for calibration, verification and demonstration purposes. The amplified signal and the artificially generated step-function were then sent to the MFAM 2.

The input signal was gated and low-pass filtered to avoid, channel interferences, distortion of the MR signals, and noise otherwise affecting measurements in inverse proportion to the short sample period of the sample-hold circuit. Any of the eight channels of the used MFAM can be individually switched to transmit different artificially generated step-function signals rather than measured signals. These signals generated internally in the modulator box were used for calibration, validation and demonstration purposes, but could also be used to verify, e.g., EPI timing. The channel clock and the filtered signals were fed to the amplitude modulators. The outputs from these were joined in a power combiner and sent to the RF output. The transmitter power was approximately 10 nW per channel. The aerial was a simple quarter wavelength flexible wire extending horizontally from the modulator box.

Images acquired while the modulator is active in the scanner room appear normal except for patterns along lines orthogonal to the frequency encoding (readout) direction. Depending on the chosen frequencies, these lines may only be visible when the total bandwidth is increased. With appropriate adjustment, the image of the scanned subject is undistorted.

Image acquisition using echo planar imaging involves high bandwidth sampling, typically around 100 kHz for approximately 100 ms. Often, the sampling period is split into shorter periods, one per line of the image. In contrast to the MR signals, the signals emitted from the MFAM are not directly influenced by changing magnetic fields. With the chosen encoding (amplitude modulation at distinct frequencies), the encoded signals can consequently be extracted from columns of a spectrogram of the measured data. It is simple and fast to calculate a spectrogram for each image, i.e., the frequency content as a function of time. This is most easily done by organizing the measured data in a matrix similar to the k-space data matrix used for image reconstruction, but with time along both axis (“true” time, and time from last beginning of a sample period). For the case of EPI, this matrix is identical to the k-space data matrix except for a reversal of every second line, and possibly an apodization. In order to calculate the spectrogram, the matrix is Fourier transformed along the “short” time axis.

The squares of the offset and encoded signals were reconstructed with time resolution equal to the EPI echo spacing by averaging of a small number of neighbouring columns of the squared absolute spectrogram. From these, the offset and scaled signals were recovered. Comparison to the simultaneously acquired known calibration signals can establish the original signal including sign and magnitude.

This analysis was done on a separate PC using locally developed software implemented in the programming language IDL (Research Systems Inc., Boulder, Colo., USA). The raw MR data and acquisition parameters were automatically saved on the MR acquisition computer and were copied via network to the separate PC by manually issuing a single copy command. The implemented software extracts a given number of embedded signals from the EPI raw data, and performs basic analysis. The timing information was derived from the acquisition parameters. The carrier frequencies were derived automatically from the raw data itself by analysis of the power profile averaged over all EPI readouts. The carriers may not have the most power, but since they give rise to sharp peaks in the spectral distribution, they were consistently found to have the most pronounced power difference compared to the neighbouring frequencies (pixels). Consequently, the carrier frequencies were detected as the distinct positions with the smallest second derivatives (negative) of the averaged power profile. The frequency range used for integration was not critical, but was chosen ±6 kHz (3 pixels on either side of the detected peak).

For the purpose of demonstrating the present invention technique, a simple and illustrative MRI protocol and setup was chosen. The gradient echo product sequence of the Siemens Trio®, 3 Tesla, whole-body imaging system was used for repeated echo planar imaging of three axial slices through the eye region of a healthy volunteer for 28 seconds. The standard quadrature head coil was used. In the middle third of the period, the subject paused with open eyes, whereas the remaining time was spent alternately looking right and left self-paced. This activity would normally be visible on both MR images and EOG, and the correlation between the two can therefore be used to probe the validity of the approach according to the invention.

Electrophysiological signals were therefore measured during MRI with MR compatible electrodes positioned near the heart and eye musculature to record ECG and EOG simultaneously.

The MRI parameters were as follows: Slice thickness 5 mm with 1 mm inter-slice gap. The echo and repetition times, TE/TR=41 ms/235 ms, were chosen minimal for the used 128×128 image matrix and 400 mm quadratic field of view (3 mm is a typical fMRI in-plane resolution). Oversampling in the readout-direction is automatically employed by the used scanner, thus providing extra bandwidth (only reflected in the raw data) for the encoded EPH-signals. Each EPI gradient lobe involved ramping up for 130 μs, a 300 μs plateau, and 130 μs ramping down. Sampling was performed in the middle 512 μs period, thus including 212 μs ramp-sampling, being unproblematic only due to the used gradient-activity triggering. The used parameters are representative of studies where emphasis is put on fMRI performance, i.e., the gradient artifacts in EPH-recordings are not minimized by sacrificing temporal or spatial resolution.

The EPI echo spacing was 0.56 ms which is therefore also the time resolution of the EPH measured during single-image acquisition when reconstructed as described above. Between image acquisitions, however, there are periods of excitation, spoiling and other acquisition pauses. With the used imaging parameters, there are 5 ms periods between EPI of neighbouring slices, where the EPH-signal was not measured. For a time domain-analysis, this would normally not require attention, but it must be considered if, e.g., the alpha-power of an EEG is to be estimated (discussed later).

In addition to the 128 readouts used for reconstruction of each EPI image, the used sequence acquires 3 reference lines for the purpose of ghost-correction. These are not phase-encoded. They contain encoded EPH-signals but were discarded from the EPH-signal reconstruction to avoid potential transients in the beginning of the EPI echo-train. The non-sampled interval therefore increased from 5 to 6.6 ms corresponding to EPH-signals being measured in roughly 90% of the available time.

Due to eddy-currents and other differences between sampling on positive and negative gradient lobes (sources of normal EPI-ghosting), modulation of every second EPH-sample is expected, but easily removed by filtering or by sacrificing half the bandwidth. The latter approach was chosen here, as the resulting Nyquist frequency, 446 Hz, is still sufficient for sampling of all EPH. Neighbouring samples were simply averaged giving a time resolution of 2×0.56 ms=1.12 ms except in the above-mentioned pauses between slices.

There may be additional signal variation with the EPI line number, e.g., from transient eddy currents from phase-encoding or slice selection gradients. This non-random noise can be estimated and filtered relatively easily but this was not necessary for the present application. It is likely to be necessary for EEG recordings.

To avoid possible modulation with the slice number, e.g., coming from MR signal leakage into the EPH-signal region, the data were filtered in the following way: The extracted EPH-signals were averaged over each image, so the time resolution was temporarily decreased from 0.56 ms to TR/3=78 ms equidistant sampling. With one sample per image, it is simple to remove oscillation in synchrony with the slice excitation by use of a filter exchanging the spectral content at frequencies ±1/(TR) with the means of the neighbouring frequencies differing by the reciprocal of the total scanning time. Subsequently, the low-frequency part of the original high-resolution time series was exchanged with the filtered version, thus removing slice selection effects occurring at single sharp frequencies in a simple and effective way. For recording of rhythmic electrophysiological signals for short periods of time, the timing or the filtering approach may need to be reconsidered, if modulation is observed.

The MR images were reconstructed using 2D Fourier transformation after Gaussian smoothing in the readout direction (2 pixel full width at half maximum, sometimes used in MRI analysis). This suppresses phase discontinuities at the edges of the acquisition matrix, thus effectively preventing side lobes of the encoded signal to leak into the MRI object region. Alternatively, the additional RF signal could be estimated and subtracted relatively easily, as it has near constant amplitude in each sub-millisecond readout period.

The positioning of the amplifier and modulator in the MR room proved to be easy and non-critical. Several positions were used, and no special attention to the orientation or positioning of the antenna was needed, although those parameters influence the amplitude of the measured non-MR signals. As the signals are influenced equally, the sampling of the known calibration signal provides a reference that can be used to make quantitative measurements.

The simple data extraction algorithm worked reliably, even when RF carriers were weak, or when they were overlapping with MR frequencies (in which case, of course, both MR images and EPH-recordings are corrupted). Consequently, for shorter acquisition periods (less than a minute) the MR images and the EPH-recordings could be viewed and correlated seconds after the acquisitions were finished with the number of EPH-signals being the only parameters needing to be manually specified. For longer acquisitions, the network copying was the most time-consuming step. The following graphs are output from the software.

FIG. 11 shows simultaneous acquisition of EPI image and the three signals (ECG, EOG and calibration) transmitted wirelessly to the scanner. The feature in the middle of the image is a transversal brain slice through the eyes. Patterns appearing left and right reflect the encoded electrical signals. The symmetric appearance comes from every second line in k-space being acquired with opposite time ordering, thus resulting in a reversal of the frequency axis. Oversampling is employed in the readout direction but the image is calculated from the raw data before truncation of the FOV so the width is twice the height in contrast to the quadratic images reconstructed by the scanner. Geometrical distortions due to the air-filled sinuses and nasal cavities are seen near the eyes. Nyquist N/2-ghosts being faint copies of the water image, are visible above and below this. The ghosts result from the use of a rudimentary, conventional image reconstruction consisting of only 2D Fourier transformation of the raw data matrix (i.e. no ghost-correction).

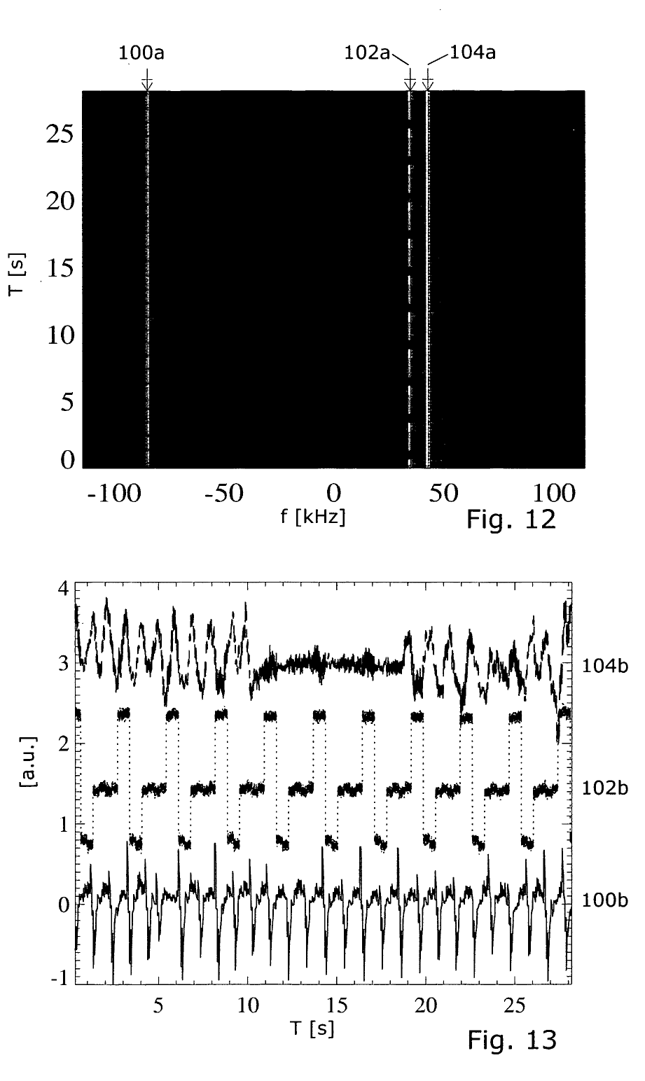

FIG. 12 shows same kind of data as FIG. 11, but reconstructed differently to reveal the time evolution of the embedded EPH and calibration signals. Starting from the raw data matrix that was Fourier transformed to give FIG. 11, every second horizontal line was here reversed to switch from a (kₓ, k_{y}) data representation to a representation with time along both axis (tₛₕₒᵣₜ, t_{long}) as k_{y} (the “blip” direction) is proportional to time for echo planar imaging. The shown image result from a subsequent 1D Fourier transformation along the short time axis, thus providing a spectrogram with frequency along the horizontal axis. In this representation, the MR signal present in the central 50 kHz region is too faint to be seen. The embedded signals, however, appear as stripes 100a (ECG), 102a (calibration) and 104a (EOG) as they are transmitted via amplitude modulated carriers at distinct frequencies. The changing amplitude is seen as intensity variations (the EOG has too little amplitude variation for this to be clearly visible).

The spectrogram of FIG. 12 is calculated from all the EPI line readouts, the raw data from the whole 28 second acquisition enters the graph, so the data shown in FIG. 11 acquired over a fraction of a second, appears over a tiny fraction of the time axis. Arrows over the graph show the position of the carrier frequencies of the ECG (100a), calibration (102a) and EOG (104a) signals. The horizontal lines across the arrows indicate the width over which the power was integrated to reconstruct the EPH-signals shown in subsequent, corresponding graphs 100b, 102b and 104b of FIG. 13. Except for holes of several milliseconds appearing near EPI excitations, the spectrogram has millisecond time resolution.

FIG. 13 shows the time courses of ECG (100b), calibration (102b) and EOG (104b) signals extracted from the MRI raw data, i.e. another presentation of the data shown in the spectrogram of FIG. 12. Graph 100b shows the beating of the heart. The high field gives rise to distortions in this ECG, but apart from this, the quality is high, considering that this is virtually unfiltered and was acquired simultaneously with high speed MR imaging. Graph 102b is the calibration signal that consists of superimposed step functions with small and large amplitude. Graph 104b is the EOG showing large oscillations in the two periods with eye motion and little variation in between. Again, the quality is good considering the extreme circumstances. The local variance of the EOG correlates with the amplitude of the calibration signal, showing that channel cross talk is a limiting factor in the current implementation. This is clearly visible only in the EOG during the middle period, but is confirmed by examining a high pass filtered version of the EOG reflecting very clearly the immediate amplitude of the calibration signal (not shown). The time resolution in the graphs of FIG. 13 is 1.12 ms except for 6.6 ms periods between EPI of neighbouring slices.

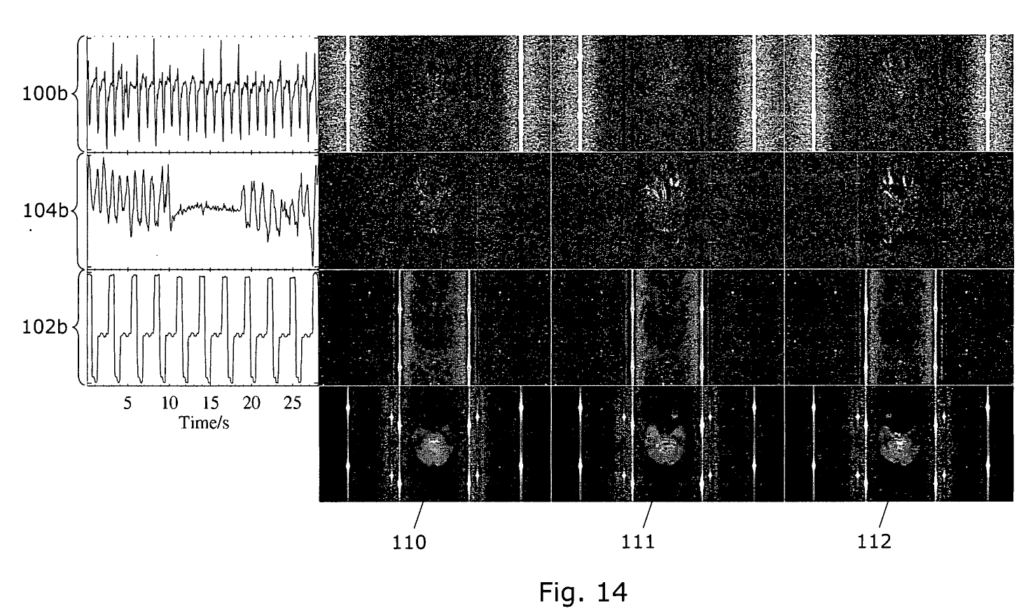

FIG. 14 shows a matrix of cross correlation maps between the measured time courses (ECG, EOG and calibration curves, left) and the corresponding temporal variation in voxel intensities for three different anatomic slices, symbolized by frames 110-112. The maps appear bright where there is high absolute correlation at zero delay, i.e. in the image regions where the signals are encoded, and in regions where the MR signal is correlated to the EPH-signals (e.g. the eye region where voxel intensities are correlated to the EOG). Also edges of the head appear bright as the head involuntarily follows the eye motion. The correlation maps are scaled differently to highlight the physiological content.

High correlation between the EOG and the image intensity is found in the eye region, and similarly, correlation is seen between the ECG and the image intensity near the medial cerebral artery and the circle of Willis, demonstrating the validity of the approach. High correlation is also found in other regions, most notably outside the head, as the signals are encoded as artifacts there. The signals are seen to take up more bandwidth than needed and the overall noise in the MR images is therefore increased. In contrast to FIG. 13, the time courses shown are averaged over each slice-acquisition period. They therefore have higher signal-to-noise ratio.

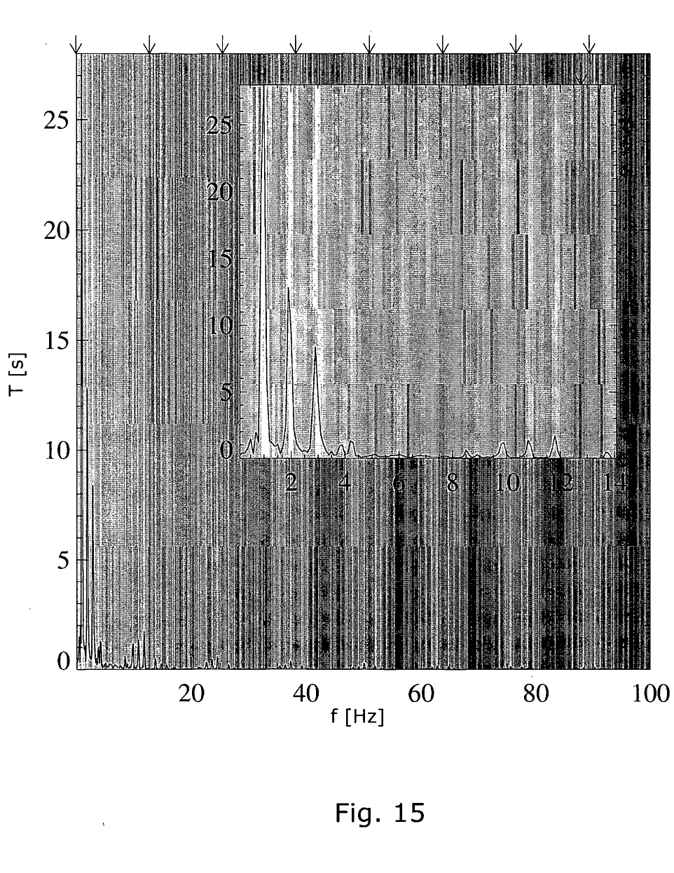

The physiological signals are measured via MR imaging and their sampling is therefore normally non-equidistant. Appropriate methods must be used for calculating the spectrogram. In the graph of FIG. 15, a Lomb-Scargle periodogram is calculated from sampling of the heart rhythm similar to graph 100b of FIG. 13. The inset graph shows a zoom of the spectral region up to 14 Hz. In this, the spectral distribution for the whole period (solid line) clearly shows that the heart frequency (˜1 Hz) and higher harmonics of this, is present in the signal. The background greyscale colour shows the amplitude of the spectrogram using a logarithmic colour scale. Artifactual spectral density appears aliased around the image repetition frequency, and multiples of this, as indicated with arrows above the graphs. Bayesian methods, for example, may be used to address this issue, which can also be solved in many situations, simply by adjusting measurement parameters.

1. A method for generating, transmitting and recording a non-magnetic resonance (non-MR) data signal during a magnetic resonance (MR) sequence, the method using a MR apparatus in a MR room, the method comprising the steps of:
in the MR room, generating the non-MR data signal and modulating the non-MR data signal onto a magnetic, electric or electromagnetic signal to form a non-MR modulated carrier signal having a frequency comprised by one or more channels of the MR apparatus,
using the MR apparatus for acquiring the non-MR modulated carrier signal by introducing the modulated carrier signal into a MR signal path of the MR-apparatus before analogue to digital conversion,
performing a MR sequence using the MR apparatus simultaneously with acquiring the non-MR modulated carrier signal by the MR apparatus, and
demodulating the introduced modulated carrier signal to restore the non-MR data signal or a function thereof,
wherein the carrier signal has a frequency which is distinguishable from frequencies of MR signals recorded in the sequence.

2. The method according to claim 1, wherein the step of generating the non-MR data signal comprises generating an electric non-MR data signal.

3. The method according to claim 1, wherein the step of modulating the non-MR data signal onto the magnetic, electric or electromagnetic signal comprises the step of generating an electric or electromagnetic carrier signal in an oscillator and modulating the non-MR data signal onto the carrier signal.

4. (canceled)

5. The method according to claim 1, wherein the frequency of the carrier signal is in an oversampled frequency range corresponding to positions outside or in the outskirts of a MR image or spectrum formed from the MR signals.

6. The method according to claim 1, further comprising the step of gating the modulated carrier signal with a magnetic field change sensitive device triggered by gradient switching.

7. The method according to claim 1, further comprising the step of recording the non-MR data signal in synchronisation with a recording of MR data obtained during the MR sequence.

8. A method for obtaining a physiological signal of a subject positioned in a magnetic resonance scanning room during a magnetic resonance scanning sequence, the method using a magnetic resonance (MR) scanner, the method comprising the steps of:
sensing and/or monitoring a non-magnetic resonance (non-MR) physiological signal of the subject,
modulating the physiological signal onto an electric or electromagnetic carrier signal having a frequency comprised by one or more channels of the magnetic resonance scanner,
transmitting the modulated carrier signal to the magnetic resonance scanner,
receiving the modulated carrier signal at the magnetic resonance scanner,
digitizing the modulated carrier signal together with MR signals recorded during the sequence, and
demodulating the modulated carrier signal to restore the physiological signal or a function thereof.

9. The method according to claim 8, wherein the step of modulating the physiological signal onto the carrier signal comprises the steps of
transmitting the sensed and/or monitored physiological signal to an RF modulator,
generating an electric or electromagnetic carrier signal in an oscillator, and
modulating the non-MR data signal onto the carrier signal in the RF modulator.

10. A magnetic resonance (MR) apparatus for receiving electric or electromagnetic (EM) carrier signals modulated with non-MR data during a MR sequence and restoring the non-MR data, the MR apparatus comprising software for accessing received and digitised MR signals and non-MR data modulated carrier signals and, using knowledge of a carrier frequency of the non-MR data modulated signals, identifying non-MR data modulated carrier signals and, using knowledge of the modulation of said non-MR data modulated signals, demodulating identified non-MR data modulated carrier signals and restoring the non-MR data or a function thereof.

11. The MR apparatus according to claim 10, further comprising software for synchronising a time stamp on the restored non-MR data signals with a time stamp on corresponding MR signals.

12. A system for recording non-magnetic resonance (non-MR) data generated inside a MR measurement room, the system comprising a modulator to be positioned inside the MR measurement room and a MR apparatus, the modulator comprising
a input part for receiving the non-MR data,
a modulating part for modulating the received non-MR data onto a magnetic, electric or electromagnetic signal to form a modulated carrier signal having a frequency comprised by one or more channels of the magnetic resonance apparatus,
the MR apparatus being adapted to receive modulated carrier signals from the modulator and transmit the received signals along a signal path for MR signals, the MR apparatus comprising software for identifying and demodulating the modulated carrier signals received from the modulator to restore the non-MR data.

13. The system according to claim 12, wherein the modulator further comprises an oscillator for generating an electric or electromagnetic carrier signal and a transmitting part for transmitting said modulated carrier signal to the MR apparatus.

14. A coil for a magnetic resonance (MR) apparatus, the coil being configured to receive and introduce electromagnetic, radio frequency (RF) MR signals and electric non-MR data signals into a signal path of a MR apparatus, the coil comprising
a receiving coil part for receiving electromagnetic RF MR signals, generating corresponding electric RF MR signals,
a socket part for receiving electric non-MR data signals,
a modulator electronically connected to the socket part, the modulator comprising
an oscillator for generating an electric carrier signal having a frequency comprised by one or more channels of the magnetic resonance apparatus, and
a modulating part for modulating received electric non-MR data signals to the carrier signal; and
one or more electric connections from the receiving coil part and the modulator to the MR apparatus for introducing the electric RF MR signals and the modulated carrier signals into the signal path of the MR apparatus.

15. A method of using a magnetic resonance apparatus comprising:
receiving non-magnetic resonance (non-MR) data modulated carrier signals inside a magnetic resonance (MR) room during a MR sequence;
digitizing the non-MR data modulated carrier signals:
transmitting the non-MR data modulated carrier signals from inside the MR room to outside the MR room;
identifying the non-MR data modulated carrier signals; and
demodulating the non-MR data modulated carrier signals.

16. The method according to claim 15, further comprising gating the non-MR data modulated carrier signals in predetermined periods of a MR sequence.

17. The method according to claim 15, further comprising synchronizing a recording of the non-MR data modulated carrier signals with predetermined periods of the MR sequence.

18. A method of using a modulator to receive and modulate non-magnetic resonance (non-MR) data generated inside a scanner room onto a magnetic, electric or electromagnetic signal to form a modulated carrier signal and transmitting the modulated carrier signal to a magnetic resonance apparatus, the modulator being positioned inside a magnetic resonance apparatus room and operating during a magnetic resonance sequence, the modulator comprising:
an input part for receiving non-MR data,
a modulating part for modulating the received non-MR data onto a magnetic, electric or electromagnetic signal to form a modulated carrier signal having a frequency comprised by one or more channels of the magnetic resonance apparatus; and
a transmitting part for transmitting the modulated carrier signal to the magnetic resonance apparatus.

19. A method for generating, transmitting and recording a non-magnetic resonance (non-MR) data signal during a magnetic resonance (MR) sequence, the method using a MR apparatus in a MR room, the method comprising the steps of:
in the MR room, generating the non-MR data signal and modulating the non-MR data signal onto a magnetic field in a reception region of a receiving coil of the MR apparatus to form a non-MR data modulated MR signal recorded by one or more channels of the MR apparatus,
using the MR apparatus for acquiring the non-MR data modulated MR signal by introducing the non-MR data modulated MR signal into a MR signal path of the MR-apparatus before an analogue to digital conversion; and
demodulating the introduced non-MR data modulated MR signal to restore the non-MR data signal or a function thereof.
